(19) United States
(12) Patent Application Publication
(10) Pub. No.: US 2020/0394737 A1
(43) Pub. Date: Dec. 17, 2020

(54) CONDITION SCREENING AND MATCHING FOR SPECIALISTS

(71) Applicant: Snapclarity Inc., Kanata (CA)

(72) Inventors: Terri STOREY, Stittsville (CA); Jennifer THAKE, Kemptville (CA)

(21) Appl. No.: 16/899,768

(22) Filed: Jun. 12, 2020

Related U.S. Application Data
(60) Provisional application No. 62/861,051, filed on Jun. 13, 2019.

(51) Int. Cl. G06Q 50/22; G16H 10/20; G16H 50/30; G16H 80/00

(52) U.S. Cl. CPC G06Q 50/22; G16H 80/00; G16H 50/30; G16H 10/20

(57) ABSTRACT

Systems and methods relating to the assessment of risks for a user relative to conditions and/or disorders. A system provides a user with a questionnaire and, based on the user responses, the user's risk for psychological conditions is assessed or estimated. If the user is at risk for any of such conditions or disorders, the severity of the risk of these disorders is assessed and a database of professional specialists is consulted. Based on the severity of the risk for these disorders, one or more professional specialists are selected based on their availability and/or fields of specialty. The user can then select one or more of these professional specialists and, once selected, the user can then arrange for a session with a selected professional specialist. The session can be conducted using the systems communications module. The system of the invention can be incorporated into a larger health care monitoring/wellness provision service.

### RELATED APPLICATIONS

This application is a non-provisional patent application that claims the benefit of U.S. provisional application No. 62/861,051 filed on Jun. 13, 2019.

### TECHNICAL FIELD

The present invention relates to matching users with professional specialists for mental and social care. More specifically, the present invention relates to systems and methods that match users with professional specialists based on results from an assessment of the users' state derived from answers provided by the users. The present invention can be used as part of an overall wellness strategy and as part of a health and wellness provision service.

### BACKGROUND

The digital revolution of the late 20th and early 21st century has transformed daily life throughout the world. Communications are now near instantaneous, data processing has improved by leaps and bounds, and the impact of the Internet is near incalculable. However, such technical advancements have not provided sufficient advantages to some health care sectors. As an example, a patient who may need to see a medical specialist still needs to pass through a number of discrete steps before access to such a specialist is given.

In one scenario, such a patient may visit his or her family doctor as a starting point.

That family doctor then needs to provide a referral to a specialist who will assess the patient's condition to determine whether an even more specialized medical professional is required. For this scenario, the process can require multiple visits to multiple doctors and professionals. In some cases, a patient does not see a specialist until months after an initial assessment from the family doctor. As can be imagined, the patient's condition may deteriorate, to the patient's detriment.

The above situation is even worse when it comes to mental health as most health professionals may not be equipped to provide a suitable assessment as to the patient's condition. Compounding the situation is the small number of qualified therapists equipped to deal with the multitude of potential conditions or disorders that the patient may suffer from.

What is therefore needed is a quick, accessible method and means for determining risks for conditions based on an assessment of a patient's self-identified symptoms and, based on that assessment, access to an available professional specialist who is equipped and trained to deal with the patient's possible condition. Preferably, such a development would provide users with options as to the professional specialist to engage while also providing the professional specialist the freedom to accept or reject incoming patients. In addition, such a means may allow the user to select professional specialists based on location, gender, and/or costs. Finally, such means may also facilitate access to the professional specialist with minimal need for physical travel unless absolutely necessary.

### SUMMARY

The present invention provides systems and methods relating to the assessment of risks for a user relative to conditions and/or disorders. A system provides a user with a specially curated questionnaire and, based on the user responses, the user's risk for psychological conditions is assessed. If the user is at risk for any of such conditions or disorders, the severity of the risk of these disorders is assessed and a database of professional specialists is consulted. Based on the severity of the risk for these disorders, one or more professional specialists are selected based on their availability and/or fields of specialty. The user can then select one or more of these professional specialists and, once selected, the user can then arrange for a session with a selected professional specialist. The session can be conducted using the systems communications module.

In other words, one implementation provides a system where clients/users will initially be asked to complete a mental health assessment that will inform clients of their level of risk for 13 commonly diagnosed mental health disorders. This assessment is based on the DSM-5 and takes approximately 10-15 minutes to complete. Clients will have immediate access to the assessments results as well as a comprehensive and individualized treatment plan delivered in real time via the platform. This treatment plan provides clients with self-help, psychosocial, and pharmacological treatment options for their level of risk based on the most recent empirical research and best practice standards. The application will continue to prompt the client to retake the assessment in order to gauge change over time and to validate the effectiveness of therapy/self-help care provision. These longitudinal data are essential for quality assurance and optimization of care.

Clients/users will have immediate access to self-help tools and peer support via a chat environment based on their presenting concerns (i.e., all within the platform of the invention). The chat space is monitored by psychotherapists who can intervene if unhelpful or potentially harmful content is perpetuated. Self-help and self-management tools are those that have been found to be effective for reducing symptoms of mood and anxiety disorders. These include CBT (e.g., goal setting, thought diaries, exposure exercises), mindfulness and self-compassion exercises, along with suggested health behaviour interventions (e.g., sleep hygiene, exercise).

After completing the mental health assessment, the members of a subscription service that uses the present invention may now access a multitude of wellness services. Concierge services are included, in which plan members are efficiently connected to an in-house nurse advocacy team, to proactively match members to programs that meet their needs. Nurse care specialists offer a level of high standards of care that is necessary to optimize the wellness strategy for members. As an example, the nurse team will guide the plan member/user toward accessing available self-screening tools to understand their health risks. This facilitates early detection of mental health concerns and proactively addresses potential risk factors. In addition, the nurses will assist plan members in accessing educational materials that promote additional learning about important aspects of risk factors. Importantly, to increase follow-through on treatment plans and help coordinate care, the nurses provide case management services to ensure plan members are efficiently connected with the right care.

In one aspect, the present invention provides a system for matching a user with professional specialists, the system comprising:
a database module containing profiles of said professional specialists;
an automated assessment module for producing an assessment of said user's risk of multiple possible conditions based on answers provided by said user to a questionnaire provided by said assessment module;
a matching module for selecting at least one professional specialist based on said assessment of said user's risks and on said profiles of said professional specialists;
   wherein
said assessment provides an estimate of a severity of risk for each of said multiple possible conditions for said user;
said assessment module provides said questionnaire to said user by way of a digital user interface on a computing device connected to said system by way of a digital communications network.

### BRIEF DESCRIPTION OF THE DRAWINGS

The embodiments of the present invention will now be described by reference to the following figures, in which identical reference numerals in different figures indicate identical elements and in which:

FIG. 1 is a block diagram of a system according to one aspect of the present invention.

### DETAILED DESCRIPTION

The present invention relates to systems and methods for matching users with professional specialists for mental and social care. This matching forms the first entry point to personalized care as it establishes a biomarker baseline for each user's mental health state as well as the user's ongoing risk levels. In one aspect, users are matched with professional specialists based on results from an assessment of the users' state derived from answers provided by the users. The present invention further provides contextual data derived from a variety of data sets and biomarkers, also known as mental health metrics that are continuously collected and interpreted in real-time and over time to create better healthcare habits for the user. These biomarkers form the basis of the users' symptomatology as it provides the necessary quantifiable data to aide in predictive health related outcomes as collected by means of the mental health assessment, AI technology, and digital devices such as wearable technology. This creates more a personalized care for the user. The present invention can be used as part of an overall wellness strategy and as part of a health and wellness provision service.

The assessment component of the present invention provides curated and specifically crafted assessment methods to users by way of a digital platform and, through the user answers to the questions, an assessment of a risk level of the user's potential psychological conditions or disorders. In addition, the present invention provides an estimation of the severity of that user's risk for certain conditions or disorders, again based on the user answers to the questionnaire. Based on the assessment and on the estimate of the user's risk for conditions, at least one professional specialist is selected for the user and details regarding these selected professional specialists are provided to the user using the digital platform. The user can then choose a selected professional specialist and can set up a virtual appointment with that selected professional specialist. When the appointment comes due, the digital platform can provide a venue by which the selected professional specialist and the user can communicate with one another for a suitable therapy session. In one implementation, the assessment method can take the form of a carefully curated questionnaire based on well-established and well-accepted practices and methodologies.

In one implementation, the professional specialist is a registered psychotherapist, social worker, or clinical psychologist suited to provide therapy and/or advice to patients/users. For this implementation, the user's risk for one or more of a multiple number of psychological conditions or disorders is assessed/estimated simply by way of answers from the user. The psychological condition or disorder for which a user may be at risk for may be one of a number of common conditions or disorders for which potential treatment may include therapy sessions with licensed therapists. It should, however, be clear that other implementations may have the user funnelled to a medical professional, such as a registered nurse, after the user's state has been assessed. The medical professional can then double check to ensure that the assessment is correct, or the medical professional can act as the gatekeeper to the system by acting as a referral to the professional specialist.

In one implementation, the digital platform is an application or app suitable for execution and/or installation on a portable data processing device such as a tablet computer or a smartphone. The assessment method is provided to the user by way of the device's user interface and can be provided to the user as a questionnaire that is given as only one question at a time. The questionnaire may be structured so that specific sections or portions of the questionnaire are designed to prompt, evoke, or draw out responses relating to a specific psychological condition or disorder. Each of these sections targeting a specific condition may also be structured such that general questions are asked first so that user responses to these early questions will determine if later, more specific questions regarding the condition in the questionnaire are even presented. As an example, if a section relates to probing the possibility of suicide, if a user responds that the ideation of suicide is not present (i.e. the user has not thought or considered it), then later, more specific questions regarding this topic may not be presented to the user. Conversely, if the user has responded that the ideation of suicide has been present, the questionnaire may present questions that probe the seriousness of the condition.

As an alternative to the above, if a questionnaire is used as the assessment method, the questionnaire may be presented in a non-structured manner, with questions about different conditions being interspersed with one another. These questions may still be configured so that responses to general questions that indicate that the condition is less likely to exist (or is negligible) in the user will cause specific questions about the condition to not be presented to the user. It should, however, be clear that each of the questions may be presented to the user with a set number of clear-cut answers so that the user can clearly understand the options available. Preferably, one of these options is one in which the user refuses to answer the question. This allows the user to be as candid or as guarded as he or she may be comfortable with. Of course, if a user refuses to answer one or more questions, a message may be displayed to the user that it is preferred that the user answer the question as the answers will provide a better assessment result as to the user's risks regarding one or more conditions.

The questionnaire may be curated or compiled based on multiple relevant sources such as journals, manuals, and other sources suitable for the purpose. In one implementation, the questionnaire may have questions that query the user's habits, feelings, issues, and thoughts. The user's answers can be the basis for further questions that delve further into more specifics about the user's actions, habits, or thoughts to determine the risks that the user may be in. Based on the user's answers, in addition to the referral or determination of a suitable professional specialist for the user, the system may provide specific advice, changes to the user's habits, or non-medicinal remedies that may assist the user with his or her state. This advice may, of course, be dependent on the system's assessment of the user's state and the amount of risk that the user may be under for a particular condition. Thus, the system may state that the user may need rest or relaxation should the user's answers show a risk of anxiety. Similarly, other life intervention-type advice may be provided by the system based on the user's state. These life intervention-type advice tips may include decreasing or discontinuing alcoholic consumption, exploring nature, reducing stress by relaxation or meditation, etc., etc. Preferably, the advice tips are specific and easily actionable by the user (i.e. easy to implement). As well, preferably, the advice tips are also culled from journals, manuals, and other material that are specific to the disorder, condition, or state that the user may be in or that the user may be at risk for. The conditions, states, or disorders that the user may be at risk for may relate to any and all types of issues regarding anxiety, sleep, stress, mental health, or any other aspect which may affect a person's well-being or sense of wellness.

Once the user has answered the questions presented, the responses are then analyzed to result in an indication of risk levels for the various conditions. This indication is a result of an appraisal that may take the form of determining which of a pre-set number of common psychological conditions or disorders that the user is at risk for. In addition, the assessment determines the severity of the risk for the condition or disorder that the user may be at risk for. This appraisal or estimation may, for every one of these common conditions, analyze the patterns of the user's responses. The analysis may take the form of determining how many of a predetermined number of indicators for a specific condition are present. Depending on the configuration of the system, a threshold for each condition may be set such that if a user has more than the threshold number of indicators, then the user is at a higher risk for that condition or conditions. Of course, severity of the risk for a condition may also be determined based on how many more indicators past the threshold are present for the user. If, as an example, there are 20 risk indicators for a given specific condition, the system may be set so that the presence of 5 or more risk indicators show the heightened risk for that condition. The presence of 5-9 of these risk indicators may indicate a mild risk for the condition. The presence of 10-14 of these risk indicators may indicate a moderate risk for the condition and the presence of 15-20 of these risk indicators may show a severe risk for the condition. It should, of course, be clear that the evaluation of the risk for a specific condition for a user and the severity of that risk is implementation dependent and may be based on a number of factors. It should also be clear that the criteria for the risk or absence of risk for a condition and the evaluation of that risk for such a condition in the user may change even within a single implementation. Furthermore, it should be clear that the estimation as to the severity of a risk for a condition is only provided as an estimate and is based mostly, if not solely, on the user responses to the questionnaire. The questions in the questionnaire may also change and may be adjusted as necessary to incorporate newer findings and research.

After the assessment of risk of a condition for a user has been made by the system according to one aspect of the invention, the risks for the user for specific conditions are ranked and/or sorted such that conditions for which the user is at most risk for are ranked highest. The conditions for which the user is at highest risk for can then be used to sort and/or select a suitable professional specialist. It should be clear that if the assessment indicates a very high risk for a specific condition and a potential emergency situation, the user can be funnelled to the first available professional specialist for immediate attention or the user can be directed to contact emergency services. The assessment results can be used as biomarkers to thereby engage and reengage users with the app and with the associated material and support networks that forms part of the invention. These biomarkers can then form objective quantifiable data that is used in the continuous assessment and diagnosis and treatment of the user's maladies.

The system according to one aspect of the invention includes a database of professional specialists. These profiles may be marked as being available or not available to denote professional specialists who are seeking new clients/patients. Each profile may include relevant data about the professional specialist such as their name, contact information, field of specialty, therapist license details, medical license (if applicable), hourly billing rate, professional biography, and potentially a photograph of the professional specialist. The profiles may be sorted by field of specialty so that it is easier to find a match between an available professional specialist (with a specific field of specialty) and the condition for which the user is at a most severe risk for. Once the sorted profiles are sorted by specialty, the system can extract the available professional specialists that have the field of specialty that is the same as the condition for which a user is at most risk for. Other criteria can then be applied to the smaller list of professional specialists with the correct field of specialty. These other criteria may include a location of the professional specialist, the gender of the professional specialist, and the billing rate or hourly rate of the professional specialist. One or more of these professional specialists can then be extracted from the list and presented to the user. Note, however, that there may be cases where there is no available professional specialist with the exact same field of specialty as the condition for which the user is at most risk for. For such cases, the profiles of the professional specialists may be sorted to determine which professional specialists have fields of specialty that are most relevant to the condition for which the user is at most risk for. These professional specialists with the most relevant fields of specialty can then be extracted from the list and presented to the user.

It should be clear that, when ordering the resulting list of professional specialists for a particular user, the list of specialists with the relevant specialty can also be sorted by the recentness of a referral or selection to the professional specialist. As an example, if 10 professional specialists have listed bipolar disorder as their field of specialty or expertise, these 10 professional specialists may be ordered such that the professional specialist who has been most recently selected by the system for another user would be at the end of the queue in the list. Similarly, the professional specialist who has had the least recent selection by the system would be placed at the front of the queue in the list. This ensures a balancing of selections from the system for the professional specialists while ensuring that the user is provided with a professional specialist with a relevant specialty.

After the system has selected at least one professional specialist for a user based on the above process (i.e. selecting the professional specialist based on the results of the assessment), the user is presented with these professional specialists and their profiles. The user can then select, based on the professional specialist profiles, which professional specialist is most useful or relevant based on the evaluation of the user's answers to the questionnaire. This may be determined by, for example, the treatment history of the professional specialist, his or her specialty, his or her length of practice, his or her location, or any other criteria selected by the user.

With a professional specialist selected, the system can then be used as a communications platform between the professional specialist and the user. The user can ask for a session with the professional specialist with the session being conducted online either through a real-time chat interface through the system or through a real-time voice/video communication. While the session may not necessarily need to be online (i.e. the professional specialist may suggest an in-person session if the user can travel), it would provide convenience for both the user and the professional specialist. It should, of course, be clear that the user, when asking for a session with the professional specialist, may need to prepay for the session. The user would, if he/she wishes to book a session, be prompted to prepay for at least the whole session with the professional specialist. The cost of the session may depend on the projected length of the session as well as the hourly rate of the professional specialist. As well, instead of paying for a single session, the user may buy blocks of time with the professional specialist. If such is the case, the system may not allow a session with a professional specialist until the user has purchased a block of time sufficient to cover a typical therapy or counselling session.

It should be clear that the professional specialist who has been matched with a specific user can opt out of taking on that specific user. Each matched professional specialist is notified of any matches with users and may have the option to review/view the users' answers to the questionnaire. The professional specialist can then, based on a review of a user's responses and on the user's profile, accept or reject the match. If the professional specialist accepts the match, then the system can schedule sessions between the professional specialist and the user. If the professional specialist rejects the match, the user may be provided with other different professional specialists. Alternatively, a matched user may be directed to an online interview process conducted by a licensed professional (e.g. a registered nurse) before the matched user is directed to his or her matched professional specialist.

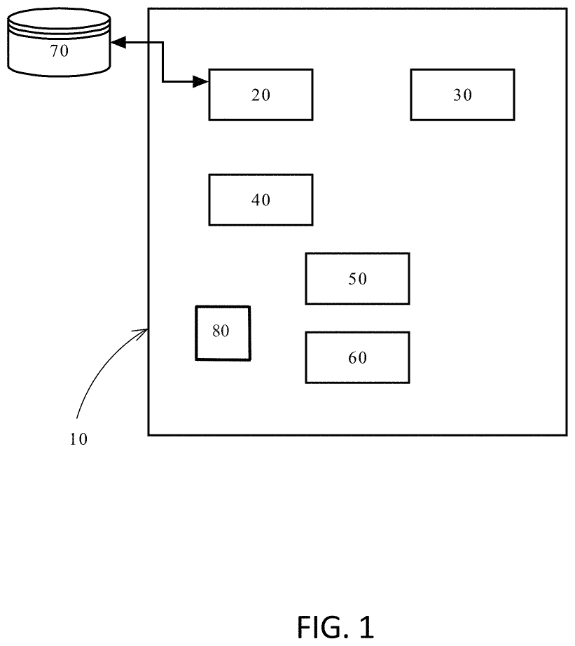

In one aspect, the present invention provides a system for accomplishing the tasks noted above. Referring to FIG. 1, a block diagram of such a system is provided. In FIG. 1, the system 10 includes a database module 20, an assessment module 30, and a matching module 40. In the implementation illustrated in FIG. 1, the system 10 also includes a communications module 50, and a payment module 60.

The database module 20 interfaces with a database 70 of profiles of professional specialists. The database module 20 may also function to filter these profiles so that only professional specialists that are currently available for new clients are provided to the system. Similarly, if the user seeking a professional specialist has any other criteria, these criteria may be applied by the database module 20 when filtering the profiles from the database. The resulting list of professional specialists that meet the various criteria can then be sent to other modules as necessary.

In one embodiment, the system also includes an automated text/voice communications module 80. This module 80 functions to provide a guided/directed automated voice/text conversation between the system and the user. The module 80 provides a text or voice interface through which predetermined questions and prompts are provided to the user along with options for predetermined answers from which the user can select a response. The module allows the user to receive prompts from the system and to provide answers to the system. The module may be used to gather information about the user as well as about the user's state. Accordingly, the module may be used to guide the user through the curated questionnaire such that the user provides responses that determine the system's view of the user's state. Similarly, the module may be used to gather additional data regarding the user should the user's responses be incomplete or inadequate to provide the system with a suitable assessment of the user's state. As well, should the user only partially complete the curated questionnaire, the module may be used to prompt the user to complete the curated questionnaire whether through the module or through a different user interface. As can be imagined, the user's inputs to the module in response to the specific prompts and/or questions are stored and can be viewed/reviewed by whichever professional specialist is dealing with the user.

In addition to the above, the communications module can include a machine learning based subsystem that probes the user for responses that indicate, whether directly, inferentially, or peripherally, the user's mental and/or physical condition. Such a subsystem can question the user and the user's responses can be assessed, in conjunction with human interpreters, to determine the user's condition. This assessment can then form, as noted above, biomarkers that would assist in the continuous, pervasive, and on-going gathering of data and diagnosis and treatment of the user. These biomarkers can be continuously assessed, and the user's condition can be continuously diagnosed and treated using the app as noted above in conjunction with assistance, advice, and diagnoses from health professionals such as registered nurses, psychotherapists, and the like. The biomarkers and the user's responses can be analyzed and contextualized in real-time by the machine learning system, in conjunction with human reviewers, to determine the proper course of treatment, advice, and diagnosis.

It should be clear that the above intelligent communications module can be configured to operate with wearable devices worn by the user so that the user's real-time vital signs can be monitored, tracked, and stored as necessary. The user's vital signs, along with the user's responses to the questions provided, can form the basis for predictive diagnoses of the user's most likely condition and the likely progression of the user's existing conditions, if any. As well, the user's responses and the user's vital signs can be the basis for one or more data sets that will be analyzed, in real-time, for up-to-date advice and guidance to help the user form healthier habits and to thereby result in a healthier lifestyle for the user.

In one implementation, the assessment and data gathering regarding the user's condition is continuous. The system can either keep asking the user questions whenever the user logs in to use the system or the system can simply re-assess the user's condition at every opportunity. This allows for a continuous monitoring of the user's condition as well as a means and opportunity to validate/adjust/reassess the treatment options/advice provided to the user. As can be imagined if the user's condition has not significantly changed after a predefined number of tries at one or more specific treatment options, the system may either flag the user's case for human intervention/review or the system may automatically adjust the treatment/advice provided to the user.

In one implementation, the module 80 may be implemented as a “chatbot” that follows a predetermined conversation path or multiple branching conversation paths for the user along a conversation decision tree. Within the conversation decision tree, each question is a node in the tree and each answer can be a gateway to its own sub-tree of options. The module may have multiple questions along with specific multiple answers provided to the user. Then, depending on which answer the user selects to which question (with the answers being provided in a multiple-choice format), the system can take different branches along the question path. Each branch, of course, may lead to further questions and answer options that have their own branches. By judiciously designing the various conversation paths in the module, the questionnaire that extracts information from the user about the user's state can be implemented using the text/voice module. Of course, instead of predetermined answers that the user simply selects from the user interface, the module may also receive typed input from the user. This typed input can then be stored for the user and can be reviewed and assessed by the specialist professional assisting the user. Conversely, the typed in answers from the user may be processed using natural language processing with artificial intelligence/machine learning to determine the user's intent.

It should be clear that, to ensure that the user's needs are uppermost, the chatbot conversations and the user's answers may be continuously monitored by one or more psychotherapists who can intervene if material that is unhelpful or harmful to the user is introduced into the chat. Similarly, if a chat between the user and another professional is in progress, this can also be monitored either by another professional or by an artificially intelligent agent that can alert other professionals if the chat indicates an escalation of a situation or if harmful consequences may result. The automated detection of such incidents when intervention may be required by another professional may be built in to the system and may be trained using suitable data sets in conjunction with machine learning.

As noted above, another implementation involves the communications module 80 as an intelligent, AI-based chatbot that continuously learns and probes the user for details about the user's condition and/or health. The user's responses can be used as data sets for training and retraining the machine learning subsystem so that it learns what are acceptable responses from the user as well as what responses are to be rejected. A feedback loop can be established so that the training and retraining is continuous and so that the subsystem learns to recognize conditions and maladies that users may have. Of course, a trained health professional can review the subsystem's performance to ensure that the module's reactions and actions are correct.

The assessment module 30 functions to present the curated assessment questionnaire (or some other assessment method) to the user when required. The questions in the questionnaire may be fixed and can be updated as necessary. The structure of the questionnaire may also be updated periodically and may be changed as necessary. As noted above, the questionnaire can be structured so that, depending on the user's responses to some of the questions, other questions may not need to be presented. Preferably, the questions are provided such that only one question at a time is presented to the user as opposed to having multiple questions presented simultaneously. Of course, in addition to presenting the questionnaire, the assessment module also receives the user's responses. These responses are analyzed by the assessment module and the assessment of the user's risk relative to one or more conditions or disorders is produced by the assessment module. This resulting assessment can then be sent to the matching module as necessary.

The matching module 40 determines which professional specialists are to be recommended to the user based on the result of the user's assessment/evaluation. The matching between the user and the professional specialists is performed by analyzing the user's assessment and the list of profiles received from the database module. Depending on the configuration, the system may query the database module for professional specialists with expertise in conditions or disorders that the user is at risk for after the user's assessment has been produced or the system may simply query the database module for a list of available professional specialists that conform to whatever criteria the user may have. Then, the matching module can filter the list from the database module for specialists with expertise in the conditions or disorders that the user is at risk for once the user's assessment has been produced. Once the matching module has determined which professional specialists are to be recommended or presented to the user, the details for these specialists are sent to the user.

It should be clear from the above that the functions of the various modules of the system are dependent on the configuration and implementation of the system. As an example, the filtering of the professional specialist profiles for those with expertise in the user's condition or disorder may be performed by the database module or by the matching module. Similarly, even though the above mentions the assessment module as producing the user's assessment, the analysis of the user's responses to the questionnaire may be performed by other modules such as, for example, the matching module.

As shown in FIG. 1, the system 10 may include a communications module 50 and a payment module 60. Depending on the implementation, these modules may be part of the system 10 or they may be external to the system. Regardless of whether these two modules form part of the system, the two modules perform useful functions for the operation of the overall system.

The communications module 50 facilitates communications between the user and one or more professional specialists. Depending on the configuration, the user may be provided with a number of options for conducting the session or consultation with the professional specialist. A video call may be provided as an option as well as an online real-time chat interface between the two participants. In addition, some implementations may also provide a text message/SMS option. However, it should be noted that the text message/SMS option is not optimal as tracking the length of the session would be difficult using this communications medium. An online chat option may be a preferred option for multiple reasons, two of which are that it as it allows for time tracking and real-time communications. A voice-only communications option (similar to a telephone voice call) may also be possible. It should be clear that the communications module interfaces with or communicates with the user's device and the professional specialist's device to set up a communications link. It should be clear that, as necessary, the results of the communications module (including any chat conversations) are monitored by psychotherapists who can intervene if unhelpful or potentially harmful content is detected in the communications.

For the payment module 60, as noted above, this may form part of the system or it may be external to the system. The payment module interfaces with the system to provide various payment options for the user as the user purchases blocks of time with a professional specialist. Alternatively, instead of purchasing blocks of session time with a specific professional specialist, the payment module may provide the user with the option of depositing a set amount of credit with the system. This credit can then be drawn upon as the user progresses through the session. Once the user has purchased a sufficient amount of time or has purchased enough credit for at least one session with a specific professional specialist, then a session can be scheduled for that specialist. Of course, since different professional specialists have different hourly rates, then this threshold may be different for different specialists. Thus, while a user may have purchased enough credit for a session with specialist A, the user may not have sufficient credit for a session with specialist B who has a higher hourly rate than specialist A. The user can then be prompted to purchase more credit if the user wishes to set up a session with specialist A.

It should, of course, be clear that the system 10 may be implemented as an application or as a software system running on a server or on a number of servers.

As noted above, the assessment may take the form of a questionnaire, but other forms of assessments of the user's condition or issue may be taken. This assessment may be continuously performed on the user whenever the user accesses the system so that the user's continuing condition is continuously assessed and monitored. Thus, the user is assessed, re-assessed and continuously monitored for changes in behaviour, habits, condition, and health. This allows for continuously adjusting the treatment provided to the user and to thereby provide suitable care as the user's condition adjusts and/or changes accordingly.

As well, the system and those operating the system and supporting the activities of the company operating the system will continuously recommend, provide, and send self-help, psychosocial, and pharmacological advice to the user to thereby change the user's habits into a more healthy, positive, and socially acceptable lifestyle. As can be imagined, the system may also incorporate artificial intelligence components so that the system can learn the user's habits and can recommend proper alternatives to habits and behaviours judged to the incongruous with the system's internal mechanisms.

It should be clear that, because the system of the present invention gathers copious amounts of data regarding the user, the user will always own that data gathered. Users can, in one implementation, view, adjust, and download the data they have provided to the system.

It should also be clear that the system of the present invention would be most useful if integrated into a health care/mental health assessment and health monitoring/advice milieu/environment. Such an environment can incorporate the system of the present invention to initially assess a user's condition, preferably based on DSM-5 and other modern acceptable materials, provide some initial advice (including self-help advice) and then incorporate the user into a continuous monitoring/care regime. This regime can include continuous assessment and reassessment of the user's condition by repeated access to the system, continuous support and care from registered health professionals, and continuous recommendations, advice, and support from health professionals. The system can also be used to provide the user with access to self-help and self-management tools and methods, such as those that have been found to be effective for reducing symptoms of mood and anxiety disorders. Such tools and assistance methods can include those that promote goal setting, thought diaries, exposure exercises, mindfulness, and self-compassion exercises, along with suggested health behaviour interventions (e.g., sleep hygiene, exercise).

The system can also be used as a gateway to members of a subscription/membership service where subscribers are given access to a multitude of wellness services. Members can avail of concierge services wherein members are efficiently connected to an in-house nurse advocacy team to proactively match members to programs that meet user needs. The nurse care specialists available to members can offer a level of high standards of care that is necessary to optimize the wellness strategy of members. In one implementation, members can be guided by the nurse team towards accessing available self-screening tools to understand the member's health risks. Such use of self-screening tools facilitates early detection of mental health concerns and proactively addresses potential risk factors. In another example, members can be assisted by nurses in accessing educational materials that promote additional learning about important aspects of risk factors. To increase follow-through on treatment plans and to help coordinate care, the nurses available to members can provide case management services to ensure members are efficiently connected with the right care.

The embodiments of the invention may be executed by a computer processor or similar device programmed in the manner of method steps or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, embodiments may be implemented in a procedural programming language (e.g.“C”) or an object-oriented language (e.g.“C++”, “java”, “PHP”, “PYTHON” or “C#”) or in any other suitable programming language (e.g. “Go”, “Dart”, “Ada”, “Bash”, etc.). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for matching a user with professional specialists, the system comprising:
a database module containing profiles of said professional specialists;
an automated assessment module for producing an assessment of said user's risk of multiple possible conditions based on answers provided by said user to a questionnaire provided by said assessment module;
a matching module for selecting at least one professional specialist based on said assessment of said user's risks and on said profiles of said professional specialists;
wherein
said assessment provides an estimate of a severity of risk for each of said multiple possible conditions for said user;
said assessment module provides said questionnaire to said user by way of a digital user interface on a computing device connected to said system by way of a digital communications network.

2. The system according to claim 1, wherein said system determines a most severe risk for one of said multiple possible conditions from said assessment and selects a selected available professional specialist from said profiles, said selected available professional specialist having a field of specialty that corresponds to said one of said multiple possible conditions for which said user has said most severe risk.

3. The system according to claim 2, wherein said system determines said most severe risk for said one of said multiple possible conditions by processing said assessment to thereby sort said multiple possible conditions of said user according to severity of risk.

4. The system according to claim 2, wherein said system selects a selected available professional specialist by sorting profiles of available professional specialists based on fields of specialty of said available professional specialists and selecting said selected available professional specialist from profiles of available professional specialists whose fields of specialty corresponds to said one of said multiple possible conditions for which said user is at most severe risk of.

5. The system according to claim 1, wherein said system determines said most severe risk for said one of said multiple possible conditions from said assessment and selects a selected available professional specialist from said profiles, said selected available professional specialist having a field of specialty that is most relevant to said one of said multiple possible conditions for which said user is at most severe risk of.

6. The system according to claim 5, wherein said system determines said most severe one of said multiple possible conditions by processing said assessment to thereby sort said multiple possible conditions of said user according to severity of risk for said user.

7. The system according to claim 5, wherein said system selects a selected available professional specialist by sorting profiles of available professional specialists based on fields of specialty of said available professional specialists and selecting said selected available professional specialist from profiles of available professional specialists whose fields of specialty is most relevant one of said multiple possible conditions for which said user is at most severe risk of.

8. The system according to claim 1, wherein said one or more professional specialists are selected based on at least one of:
gender;
location of said professional specialist; and
billing rate of said professional specialist.

9. The system according to claim 1, wherein said professional specialists are medical professionals.

10. The system according to claim 9, wherein said profession specialists are registered psychological therapists.

11. The system according to claim 9, wherein said profession specialists are social workers.

12. The system according to claim 1, wherein said multiple possible conditions of said user are psychological disorders.

13. The system according to claim 1, wherein said system further comprises a communications module for allowing communications between said professional specialists and said user.

14. The system according to claim 1, wherein said communications module allows for real time online chat communications between said user and a selected professional specialist.

15. The system according to claim 1, wherein said communications module allows for real time video conferencing between said user and a selected professional specialist.

16. The system according to claim 1, wherein said one or more professional specialists are selected based on a recentness of a last selection for said professional specialists.

17. The system according to claim 1, wherein said profiles of said professional specialists are sorted based on how recent each professional specialist has been selected by said system.

18. The system according to claim 1, wherein said system further comprises a payment module for receiving payment from users buying time for therapy sessions with one or more professional specialists.
